(19) United States
(12) Patent Application Publication
(10) Pub. No.: US 2010/0286468 A1
(43) Pub. Date: Nov. 11, 2010

(54) TRANSCRANIAL MAGNETIC STIMULATION WITH PROTECTION OF MAGNET-ADJACENT STRUCTURES

(76) Inventors: David J Mishelevich, Playa del Rey, CA (US); M. Bret Schneider, Portola Valley, CA (US)

Correspondence Address: SHAY GLENN LLP, 2755 CAMPUS DRIVE, SUITE 210, SAN MATEO, CA, 94403, US

(21) Appl. No.: 12/680,912

(22) PCT Filed: Oct. 27, 2008
(86) PCT No.: PCT/US08/81307

§ 371 Date: Jul. 14, 2010

Related U.S. Application Data
(60) Provisional application No. 60/983,140, filed on Oct. 26, 2007.

(51) Int. Cl. A61N 2/02

(52) U.S. Cl. 600/12; 600/13

(57) ABSTRACT

System and methods for Transcranial Magnetic Stimulation (TMS) are described in which regions adjacent (e.g., to the sides and behind the TMS electromagnet) are protected from the high magnetic fields emitted by the TMS electromagnet. Thus, adjacent muscle or neural structures are protected and undesirable side effects are avoid or minimized, allowing stimulation from previously unavailable sites such as the mouth and pharynx.

### CROSS REFERENCE TO RELATED APPLICATIONS

This patent application claims priority to U.S. Provisional Patent Application Ser. No. 60/983,140, filed on Oct. 26, 2007, titled “TMS WITH PROTECTION OF MAGNET-ADJACENT STRUCTURES,” which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD OF THE INVENTION

The devices and methods described herein relate generally to delivery of magnetic fields to stimulate target brain regions using Transcranial Magnetic Stimulation while protecting adjacent or nearby non-target regions.

### BACKGROUND OF THE INVENTION

Transcranial Magnetic Stimulation (TMS) has been previously delivered from electromagnets that have been located at one or the other of the side of the head or from the top or somewhere in between the side and the top of the head. Generally speaking, a single or double standard TMS coil placed on a patient's scalp and operated at a power level at, or slightly above, a patient's motor threshold will directly active neurons from the cortical crowns to the bottom of the cortical gyri—a depth of about 1-3 cm. Using this approach, deeper structures (herein referred to as “subcortical”, even when these deeper areas are histologically layered in nature) are activated only secondarily through intracerebral neural connections. Conventional approaches typically do not reach greater depths (for example, to the cingulate gyrus, the insula and other subcortical structures). Deep brain modulation cannot be accomplished by simply turning up the power of the stimulating electromagnet, because the intervening tissue, for example superficial cortex, will be over-stimulated, causing undesired side effects such as seizures.

A device for providing deep-brain stimulation with Transcranial Magnetic Stimulation is described in Schneider and Mishelevich, U.S. patent application Ser. No. 10/821,807). In these example, multiple TMS electromagnets and/or moving TMS electromagnets may be used to accumulate stimulator effects of magnetic fields at greater depths, while protecting the intervening (e.g., more superficial) neural tissue from overstimulation or even threshold stimulation.

U.S. Pat. No. 7,153,256, US patent application 2006/0122454 A1 and a recent Davey and Riehl 2006 published reference (Davey K. R., and M. E. Riehl, “Suppressing the Surface Field During Transcranial Magnetic Stimulation,” IEEE Transactions on Biomedical Engineering, Vol. 53, No. 2, February 2006) all address alternative methods of altering magnetic field distribution so as to lessen superficial (e.g. nerves in skin) stimulation proximal to the target, while permitting deeper magnetic field loops to reach the targeted cerebral cortex. These techniques typically involve lessening field loops close the coil surface, but do not offer means for restricting the distribution of the magnetic field lateral to the target area. Also, the cited references do not provide means for limiting the strength of the field radiating from the opposite side of the magnet that faces away from the target.

It would be extremely beneficial to more effectively shape the field emanating from TMS generated magnetic fields such that the field lateral to the target and the field emanating from the reverse face of the magnet are reduced.

It would also be beneficial to place electromagnets in locations which are currently inaccessible, because of the uncomfortable and potentially deleterious effect of the magnetic fields emitted by currently available TMS systems. This locations, including facial locations or locations within the patients mouth and pharynx, potentially offer additional trajectories that were previously unavailable, while avoiding other problematic trajectories (e.g. to the brain through the eye). The methods, systems and devices described herein may therefore be used to provide these new trajectories, including stimulation via the mouth, pharynx, and other inferiorly-located positions in such a way that sensitive adjacent structures are protected from stimulation sufficiently to avoid undesirable side effects such as painful muscle contractions.

### SUMMARY OF THE INVENTION

Described herein are methods, systems and devices for modifying the magnetic field emitted by a Transcranial Magnetic Stimulation (TMS) electromagnet, so that the magnetic field emitted in one or more direction from the TMS electromagnet is reduced or eliminated. Methods of using such magnets are also described.

For example, described herein are shielding enclosures configured to partially enclose a TMS electromagnet so that the magnetic field emanating from the back (and in some variations, the sides) of the TMS electromagnet does not project into adjacent tissues. Thus, the magnetic field emanating from TMS magnetic field lateral to the target, and that from the reverse face of the magnet looking away from the target, are substantially reduced in intensity.

The shielding enclosure may channel, reflect or attenuate the magnetic field, to prevent the magnetic field from being emitted through the shielding enclosure and into non-target regions adjacent to the TMS electromagnet. Thus, the shielding enclosure may shape the magnetic field. Shielding and shaping of the magnetic field from the TMS electromagnet enables placement of powerful TMS electromagnets in new locations that were previously not usable, due to the pain and involuntary muscular contractions that would otherwise be produced. A shielded enclosure as described herein may allow TMS electromagnets to be used in locations including the interior of mouth or pharynx (e.g., the nasopharyx, laryngopharynx, etc.), making a greater number of trajectories available for use by TMS, while still avoiding problematic trajectories (e.g. to the brain through the eye). These new trajectories include stimulation from the mouth, pharynx, and other inferiorly-located positions in such a way that sensitive adjacent structures are protected from stimulation at levels that provoke undesirable side effects such as painful muscle contractions.

One or more electromagnets employed for Transcranial Magnetic Stimulation (TMS) may be positioned within a shielding enclosure. In some variations the TMS electromagnet is secured within the shielded enclosure (e.g., affixed to one or more portion. In some variations, the TMS electromagnet is removeably placed or secured in the shielding enclosure. For example, a shielding enclosure may be configured to be reusable, and exchangeable over different TMS electromagnets. In some variations, the shielded enclosure may be configured so that a variety of sizes and shapes of TMS electromagnets may be placed therein. The outside of the protective shielding enclosure may be configured to have an atraumatic surface, so that it can be positioned with a body cavity (e.g., mouth, pharynx, nasal cavity, etc.).

Thus, a TMS electromagnet within a shielding enclosure may be placed in or on a patient (e.g., in the patient's mouth or pharynx, for example) where there are sensitive adjacent structures. Thus, it is important to limit the lateral spread of current from the TMS electromagnet due to the emitted magnetic field. When the TMS electromagnet is at least partially enclosed in an shielding enclosure, the ‘emitted magnetic field’ refers to the effectively emitted magnetic field, which is emitted from the shielding enclosure, or (in some variations) in a direction perpendicular to the back and/or sides of the shielding enclosure.

For example, TMS through the mouth and/or face is normally not permissible because the powerful magnetic fields emitted would stimulate non-target tissue, such as the muscles inserting on the anterior mandible, and clause undesirable side effects, such as painful muscle contractions or stimulation of non-target neural structures. Thus, neural or neuromuscular structures adjacent to a TMS electromagnet used for such stimulation would otherwise be affected by magnetic pulses from the TMS electromagnet. These side effects may be avoided using the systems and devices described herein.

For example, a Transcranial Magnetic Stimulation (TMS) system for stimulating a patient's neuronal tissue while avoiding undesirable side effects on non-target regions adjacent to the magnet may include: a TMS electromagnet configured to apply Transcranial Magnetic Stimulation to the patient, wherein the TMS electromagnet includes a front face and a back face; and a shielding enclosure for partially enclosing the TMS electromagnet, wherein the shielding enclosure is configured to prevent or limit the emission of a magnetic field into adjacent tissue from the back face of the TMS electromagnet.

The shielding enclosure may have any appropriate shape. In general, the shielding enclosure includes an open face from which the front face of the TMS is exposed to emit the TMS magnetic field towards the target structure, and a closed back and/or sides. In some variations, the shielding enclosure comprises a back wall configured to prevent or limit emission of a magnetic field from the back face of the TMS electromagnet out of the back wall, and further wherein the shielding enclosure comprises one or more side walls configured to limit or prevent the emission of a magnetic field from the TMS electromagnet through the side walls and into adjacent tissue. For example, the shielding enclosure may include a back wall and a plurality of side walls configured to prevent or limit the emission of a magnetic field from the TMS electromagnet therethrough.

The shielding enclosure may partially enclose the front face of the TMS electromagnet. For example, forming a window of a specific size or shape, or by having side walls that shape or direct the magnetic field from the TMS electromagnet. The side walls may projects towards the front of the enclosure (e.g., extending beyond the face of the TMS electromagnet. In some variations the TMS electromagnet projects from the shielding enclosure when the TMS electromagnet is in the enclosure. In other variation, the TMS electromagnet is recessed (or partially recessed) in the TMS shielding enclosure.

The side walls of the shielding enclosure may be parallel, or may be angled. For example, the side walls of the enclosure may be angled away from each other.

In general, the shielding enclosure is made of a material that can attenuate, channel, or reflect all or some of the magnetic field emitted by the TMS electromagnet. For example, the shielding enclosure may be formed of a material having a high magnetic permeability. In some variations, the shielding enclosure is formed of a mu metal (e.g., a nickel-iron alloy, such as an alloy formed of approximately 75% nickel, 15% iron, plus copper and molybdenum) that has very high magnetic permeability. Other materials may also be used, including commercial materials such as “Giron” and “MetGlas”. In general, materials that are highly magnetically permeable may be used, as magnetic lines of force preferentially travel through permeable materials, providing a route for the magnetic lines of force. Thus, the shielding materials may give the magnetic lines of force a pathway to travel, attenuating or preventing the magnetic field from passing through the region outside or adjacent to the back and/or sides of the shielding enclosure. Although the field emitted from the TMS electromagnet still exists, it may be concentrated within the shielding enclosure walls, and the lines of force will re-appear its edges. Thus, Field density at the edges of shielding may be high; thus the exposed edges of the walls may be directed towards the front face direction (e.g., therefore directed towards the target).

The materials used to form the shielding enclosure described above are passive shields. Active shields may also be used, such as an active winding that is driven by electronics to cancel the TMS electromagnet field in the non-target direction. The shielding enclosure may not block, stop, or re-route all the magnetic field lines from a TMS electromagnet; for example, some lines of force may not be re-routed through the shielding and will appear on the other side of it, however, the shielding enclosure may attenuate the overall magnetic field passing through non-target regions adjacent (e.g., to the sides and behind) the TMS electromagnet.

In some variations, the systems described herein include a plurality of TMS electromagnets. For example, the systems described herein may be used a plurality of TMS electromagnets that may be used to achieve deep-brain stimulation (e.g., TMS of deep-brain regions). Deep-brain regions are regions typically deeper than the superficial (e.g., cortical) regions that are normally difficult to reach without stimulating (and possibly damaging) more superficially located regions, between the TMS electromagnet and the deeper target. The plurality of TMS electromagnets may be positioned, moved and otherwise controlled by a controller that coordinate the operation of the plurality of TMS electromagnets in order to stimulate a neural target, including deep-brain targets. In some variations, one or more TMS electromagnet is positioned or secured within a shielding enclosure (e.g., one enclosure per electromagnet), while other TMS electromagnets not within the shielding enclosure. In other variations, all of the TMS electromagnets in the system are within shielding enclosures.

The TMS electromagnet and shielding enclosure may be configured to be inserted into a patient's mouth or pharynx. For example, the shielding enclosure may be sized or shaped to fit within a patient's mouth or a portion of the patient's pharynx (e.g., nasopharynx).

Also described herein are Transcranial Magnetic Stimulation (TMS) systems for stimulating a patient's neuronal tissue while avoiding undesirable side effects on non-target regions adjacent to the magnet that include: a TMS electromagnet configured to apply Transcranial Magnetic Stimulation to the patient, wherein the TMS electromagnet includes a back face and a front face; and a shielding enclosure configured to partially enclose the TMS electromagnet, wherein the shielding enclosure is formed of a high magnetic permeability material, and further wherein the shielding enclosure is configured to modify the magnetic field emitted by the TMS electromagnet to attenuate the magnetic field emitted through the shielding enclosure from the back face of the TMS electromagnet; wherein the shielding enclosure and TMS electromagnet are configured to be combined and placed within a patient's mouth or pharynx.

As described above, these systems may include a plurality of TMS electromagnets, and the shielding enclosure may include a back wall configured to prevent of attenuate the emission of a magnetic field from the back face of the TMS electromagnet through the back wall, and one or more side wall configured to attenuate the emission of a magnetic field from the TMS electromagnet through the side wall(s). For example, the shielding enclosure may include a back wall and a plurality of side walls configured to attenuate the emission of a magnetic field from the TMS electromagnet therethrough.

Also described herein are methods of performing TMS on a patient using a system such as those described above. For example, described herein are methods for Transcranial Magnetic Stimulation (TMS) of a neuronal target including the steps of: positioning a shielded TMS electromagnet in a patient's mouth or pharynx, wherein the shielded TMS electromagnet comprises a TMS electromagnet partially surrounded by a shielding enclosure comprising a high magnetic permeability material configured to attenuate the magnetic field emitted through the shielding enclosure from the TMS electromagnet; and emitting an electromagnetic field from the front of the TMS electromagnet through the patient's mouth or pharynx and into the brain to stimulate a neuronal target. The method may also include positioning a second TMS electromagnet around the patient's head; an electromagnetic field may be emitted from the second TMS electromagnet to stimulate the neuronal target.

In some variations of this method, an anesthetic to numb the patient's mouth or pharynx may be applied to the patient when these methods are performed.

### BRIEF DESCRIPTION OF THE DRAWINGS

FIG. 1 is an outline of patient face showing an electromagnet in position is the mouth.

FIG. 2 is an outline of the patient face of FIG. 1 showing a neural structure/target for Transcranial Magnetic Stimulation.

FIG. 3 is a diagram of a shielding enclosure surrounding an inferiorly placed electromagnet, so that the shielding enclosure may focus the magnetic field from the TMS electromagnet and protect structures inferior to or lateral to the electromagnet.

FIG. 4A is a diagram illustrating magnetic field lines representative of a TMS electromagnet and shielding enclosure of the type of shown in FIG. 3.

FIG. 4B is a diagraph illustrating magnetic field lines representative of a TMS electromagnet and shielding enclosure similar to the type of FIG. 3, but where the high-permeability side shields are angled.

### DETAILED DESCRIPTION OF THE INVENTION

Described herein are systems and methods for Transcranial Magnetic Stimulation (TMS) using one or more shielding enclosures that may be placed at least partially over a TMS electromagnet to prevent undesirable stimulation of non-target regions behind or next to the TMS electromagnet. In general, a shielding enclosure is configured to at least partially enclose or cover a TMS electromagnet, and includes a back (and may include sides and optionally a front cover). The shielding electromagnet if formed of a material that can reflect and/or channel and/or attenuate the magnetic field emitted by the TMS electromagnet enclosed therein. The shielding enclosure may be part of a system including a plurality of electromagnets, and one or more of the electromagnet may be at least partially enclosed within the shielding enclosure.

As mentioned above, the shielding enclosure may protect adjacent patient tissues, including regions behind or next to the TMS electromagnet. This may allow the TMS electromagnet to be operated in regions not previously accessible by the TMS systems, including regions in front of the patient (e.g., the face) or within the patient's mouth or pharynx.

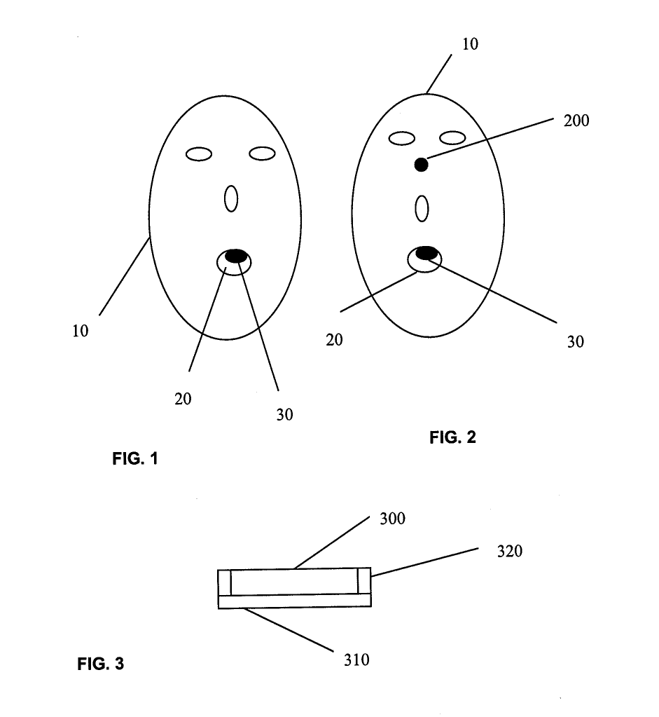

For example, FIG. 1 illustrates schematically an example in which a TMS electromagnet is placed within a patient's mouth. The patient (e.g., a person or animal) 10 has a mouth 20 in which is located magnetic source 30. The circumference of the magnet may be large enough to temporarily stretch out the cheeks. FIG. 2 shows the magnet 30 relative to a target 200, which is located within the patient's head (e.g., brain). For example, the target may be all or a portion of the cinguate gyrus, or other brain regions, particularly regions near the pharynx or mouth.

A system may be configured for stimulation of deep-brain regions. For example, the system may include a plurality of TMS electromagnets that may be coordinated (e.g., by a controller) to apply TMS to a single target from multiple sites. For example, the system may include a TMS electromagnet that is enclosed with a shielding enclosure and is placed within the patient's mouth (as shown in FIGS. 1 and 2); this system may also include one or more additional TMS electromagnets which may be positioned outside of the patient, e.g., a positions around the patient's head, for stimulating the same, or different targets as the TMS magnet within the patient's mouth.

As mentioned, a shielding enclosure typically includes a cavity into which the TMS electromagnet may be positioned, and a window through which the face of the TMS electromagnet may emit the magnetic field toward a target. The enclosure may also include channels or openings to provide the TMS electromagnet with power or the like. The outside of the enclosure may be configured for insertion into a patient's body (e.g., mouth or pharynx), particularly for non-invasive insertion. For example, the outside may be sterile or sterilizable, and may be smooth or may be shaped to conform to a region of the patient's anatomy, such as the mouth or pharynx.

The shielding enclosure may also include connections that permit the enclosure (and/or the TMS electromagnet) to be connected to the rest of a TMS system. For example, the enclosure may include attachment regions for connecting to an arm or gantry, or other positioning device. IN some variations, the outside of the enclosure is configured to be anchored to the patient. For example, the outside of the enclosure may be configured to be secured against the patient's mouth (e.g., teeth), or within the pharynx or other regions.

The shielding enclosure may also include passive or active servos or drivers for adjusting the position (e.g., the angle) of the TMS electromagnet. Passive drivers may be gears or the like for changing the angle of the TMS electromagnet (and particularly the front face of the TMS electromagnet so that it can be directed towards a target brain region. Active drivers may be powered drivers that move the TMS electromagnet. Drivers may also provide positional feedback. For example, the position or angle of a TMS electromagnet may be adjusted within the shielding enclosure, e.g., moving the TMS electromagnet relative to the shielding enclosure. In some variations, the TMS electromagnet may remain in a fixed position relative to the enclosure, and the orientation of the TMS electromagnet may be changed by changing the orientation of the entire enclosure.

FIG. 3 shows a simple cross-section through one variation of a shielding enclosure, which may direct, reflect, channel or attenuate the magnetic field in directions other than the forward (target) direction. In this embodiment, the back face (opposite the target, or in the case of mouth positioning, the inferior surface) of the electromagnet 300 can be covered with back-face shielding 310. Back-face shielding 100 may be composed of a high-permeability substance such as mu metal. Back face shielding 310 may help substantially block passage of magnetic field loops in the direction opposite the target, or in the example of mouth or pharynx placement, block in the inferior direction. As shown in FIG. 3, the enclosure may also include sides 320 that are also made from a high-permeability material such as mu-metal to reduce the amount of stimulation of adjacent structures such as nearby muscles or neural structures. Back face shielding 310 and side shielding 320 may also be formed of a single piece of suitable material.

In any of the examples described herein, the patient may also be anesthetized, so that the adjacent muscles or other structures may be anesthetized to further avoid or moderate painful contractions or undesirable effects on nerves. In this manner, if the electromagnet is placed in the mouth, the tongue, larynx, and mandibular muscles and gingiva will not receive excessive (e.g., painful) stimulation.

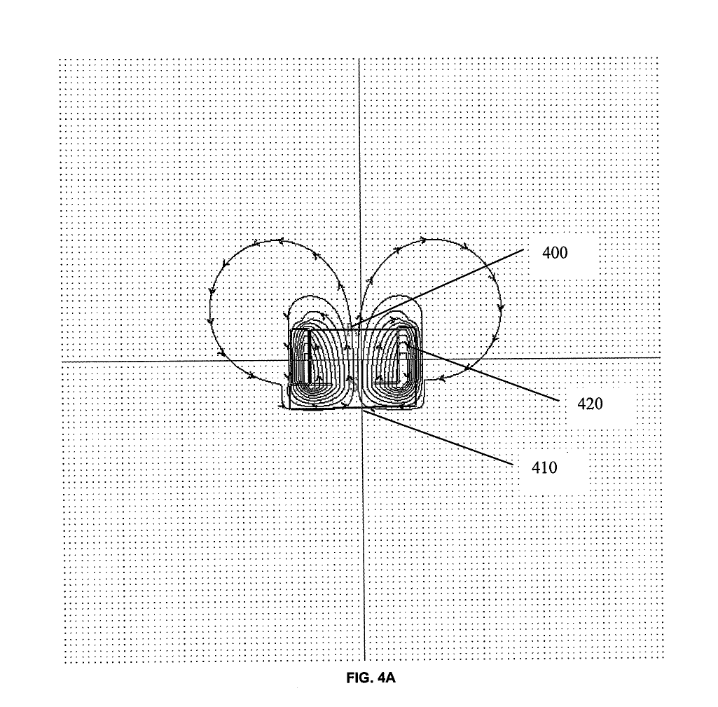

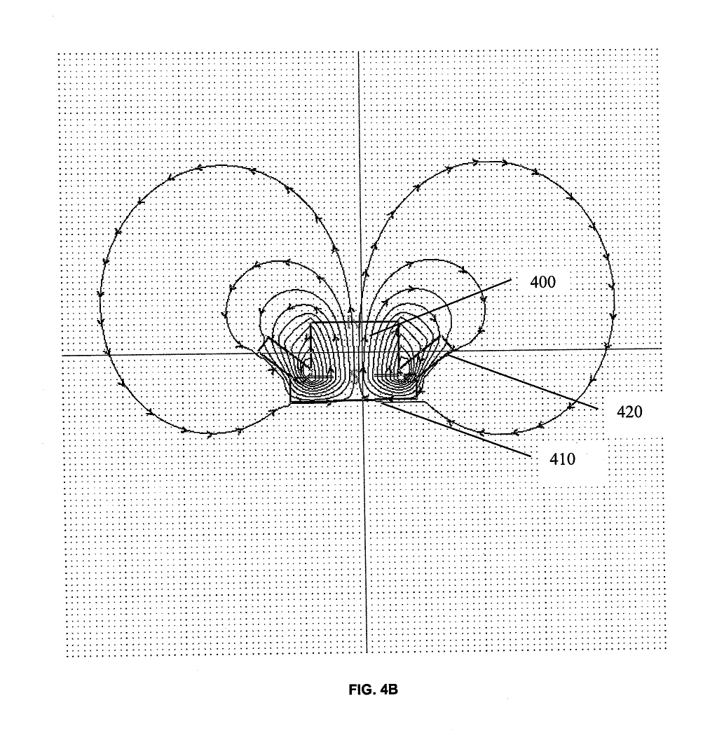

FIG. 4A is a plot of magnetic field lines related to a configuration of magnet and shielding such as the one shown in FIG. 3. Components illustrated include TMS electromagnet 400, inferior shield 410 and side shields 420. In this example, the emitted magnetic field (indicated by the magnetic force lines) is channeled by the shielding enclosure 410, 420 so that the magnetic field is emitted primarily from the front face, and can be directed specifically at a target. Although some magnetic force lines may extend beyond the shielding enclosure, in general, the magnetic field is much lower in these regions.

FIG. 4B illustrates another variation of a cross-section through a shielding enclosure showing representative magnetic force lines. In this example, the side shields 420 are angled out, opening up the magnetic field, although the region behind the enclosure is still protected.

While the embodiment described and illustrated herein are configured for use within a patient's mouth, it should be understood that a shielding enclosure may be used at any location adjacent to the electromagnet having structures that might be over-stimulated, including the pharynx, throat and other regions.

As described herein, the magnetic field emanating laterally and from the back (back face) of a TMS electromagnetic may substantially reduced in intensity by the shielding enclosure. This shaped field may thus allow placement of powerful TMS electromagnets in locations that were previously not practical, because they would result in pain and involuntary muscular contractions. Such locations may include the interior of mouth or nasopharyx.

In operation, a TMS electromagnet may be placed or secured within a shielding enclosure, and positioned so the TMS electromagnet may emit a magnetic field at a target. The TMS electromagnet may be secured or anchored within the shielding enclosure. For example, the TMS electromagnet may be permanently or temporarily secured in position. For example, a set screw may be used. In some variations the enclosure secures over the TMS electromagnet clam-shell like, so that two halves of the enclosure close over the TMS electromagnet. In other variations, the TMS electromagnet slides into the cavity formed in the enclosure. The TMS electromagnet may be removably secured within the enclosure.

The TMS electromagnet may be completely within the enclosure, or may project slightly out of the enclosure. In some variations, the shielding enclosure may include a region that extends beyond the TMS electromagnet (e.g., beyond the front face of the TMS electromagnet). In general, the sides of the enclosure maybe configured to avoid concentrating the magnetic field unnecessarily. For example, the sides may include a flat front (as illustrated in FIGS. 3-4B), or a rounded front face.

Once the TMS electromagnet is secured within the enclosure, it may be positioned relative to the patient, and then stimulated to emit a magnetic field. For example, the TMS electromagnet and enclosure may be positioned within a patient's mouth or pharynx, as illustrated. The enclosure and TMS electromagnet may then be secured (or held) in position. As mentioned, the enclosure and magnet may be secured against the patient, e.g., within the patient's mouth.

One or more additional TMS electromagnets may also be positioned around the subject's head. For example, a plurality of TMS electromagnets (including one or more that are within a shielding enclosure). Stimulation from the TMS electromagnet(s) may be controlled and/or coordinated by a controller.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention, which is set forth in the following claims.

### REFERENCES

Schneider, M. B. and D. J. Mishelevich, “Robotic apparatus for targeting and producing deep, focused transcranial magnetic stimulation,” U.S. patent application Ser. No. 10/821,807
“Trajectory-Based Transcranial Magnetic Stimulation,” Mishelevich D J and Schneider M B, Pending U.S. patent application Ser. No. 11/429,504
Rieh, M. E., and K. M. Ghiron, “Reducing discomfort caused by electrical stimulation,” US Patent Application No. US 2006/0122454 A1
Riehl, M. E and S. W. Miller, “Reducing discomfort caused by electrical stimulation,” U.S. Pat. No. 7,153,256
Davey K. R., and M. E. Riehl, “Suppressing the Surface Field During Transcranial Magnetic Stimulation,” IEEE Transactions on Biomedical Engineering, Vol. 53, No. 2, February 2006

What is claimed is:

1. A Transcranial Magnetic Stimulation (TMS) system for stimulating a patient's neuronal tissue while avoiding undesirable side effects on non-target regions adjacent to the magnet, the system comprising:
a TMS electromagnet configured to apply Transcranial Magnetic Stimulation to the patient, wherein the TMS electromagnet includes a front face and a back face; and
a shielding enclosure for partially enclosing the TMS electromagnet, wherein the shielding enclosure is configured to prevent or limit the emission of a magnetic field into adjacent tissue from the back face of the TMS electromagnet.

2. The system of claim 1, wherein the shielding enclosure comprises a back wall configured to prevent or limit emission of a magnetic field from the back face of the TMS electromagnet out of the back wall, and further wherein the shielding enclosure comprises one or more side walls configured to limit or prevent the emission of a magnetic field from the TMS electromagnet through the side walls and into adjacent tissue.

3. The system of claim 1, wherein the shielding enclosure comprise a back wall and a plurality of side walls configured to prevent or limit the emission of a magnetic field from the TMS electromagnet therethrough.

4. The system of claim 3, wherein the side walls are angled away from each other.

5. The system of claim 1, wherein the shielding enclosure is formed of a material having a high magnetic permeability.

6. The system of claim 1, wherein the shielding enclosure is formed of a mu metal.

7. The system of claim 1, further comprising a plurality of TMS electromagnets.

8. The system of claim 1, wherein the TMS electromagnet and shielding enclosure are configured to be inserted into a patient's mouth or pharynx.

9. A Transcranial Magnetic Stimulation (TMS) system for stimulating a patient's neuronal tissue while avoiding undesirable side effects on non-target regions adjacent to the magnet, the system comprising:
a TMS electromagnet configured to apply Transcranial Magnetic Stimulation to the patient, wherein the TMS electromagnet includes a back face and a front face; and
a shielding enclosure configured to partially enclose the TMS electromagnet, wherein the shielding enclosure is formed of a high magnetic permeability material, and further wherein the shielding enclosure is configured to modify the magnetic field emitted by the TMS electromagnet to attenuate the magnetic field emitted through the shielding enclosure from the back face of the TMS electromagnet;
wherein the shielding enclosure and TMS electromagnet are configured to be combined and placed within a patient's mouth or pharynx.

10. The system of claim 9, further comprising a plurality of TMS electromagnets.

11. The system of claim 9, wherein the shielding enclosure comprises a back wall configured to prevent of attenuate the emission of a magnetic field from the back face of the TMS electromagnet through the back wall, and one or more side wall configured to attenuate the emission of a magnetic field from the TMS electromagnet through the side wall(s).

12. The system of claim 1, wherein the shielding enclosure comprise a back wall and a plurality of side walls configured to attenuate the emission of a magnetic field from the TMS electromagnet therethrough.

13. The system of claim 12, wherein the side walls are angled away from each other.

14. The system of claim 9, wherein the shielding enclosure is formed of a material having a high magnetic permeability.

15. The system of claim 9, wherein the shielding enclosure is formed of a mu metal.

16. A method for Transcranial Magnetic Stimulation (TMS) of a neuronal target, the method comprising:
positioning a shielded TMS electromagnet in a patient's mouth or pharynx, wherein the shielded TMS electromagnet comprises a TMS electromagnet partially surrounded by a shielding enclosure comprising a high magnetic permeability material configured to attenuate the magnetic field emitted through the shielding enclosure from the TMS electromagnet; and
emitting an electromagnetic field from the front of the TMS electromagnet through the patient's mouth or pharynx and into the brain to stimulate a neuronal target.

17. The method of claim 16, further comprising positioning a second TMS electromagnet around the patient's head.

18. The method of claim 16, further comprising emitting an electromagnetic field from a second TMS electromagnet to stimulate the neuronal target.

19. The method of claim 16, further comprising applying an anesthetic to numb the patient's mouth or pharynx.
